(19) United States
(12) Patent Application Publication
(10) Pub. No.: US 2009/0187175 A1
(43) Pub. Date: Jul. 23, 2009

(54) Disposable Hand Piece for Dental Surgical Laser

(75) Inventors: Shlomo Assa, Valley Center, CA (US); Steven Jerome Meyer, San Diego, CA (US); Julie Assa, Valley Center, CA (US); Gordon J. Foote, San Diego, CA (US)

Correspondence Address: FISH & RICHARDSON, PC, P.O. BOX 1022, MINNEAPOLIS, MN, 55440-1022, US

(73) Assignee: INLIGHT CORPORATION, San Diego, CA (US)

(21) Appl. No.: 12/016,923

(22) Filed: Jan. 18, 2008

(51) Int. Cl. A61B 18/22

(52) U.S. Cl. 606/16

(57) ABSTRACT

A disposable hand piece can be attached at the end of a laser beam delivery assembly. The hand piece can be longitudinally tapered and generally wedge-shaped in its width and slightly bent so as to form a tool which can be comfortable grasped by a Dentist for purposes of more accurately controlling the laser beam applied through the laser hand piece. The hand piece can include an internal means to supply fine mist of air and water for cooling of the treated area. The hand piece is constructed of inexpensive plastic materials and is designed to be disposable after a single use.

### CROSS-REFERENCE TO RELATED APPLICATIONS

This application is related to U.S. patent application Ser. No. TBD, entitled LASER SURGICAL METHODS, to inventors Shlomo Assa and Steve J. Meyer, which application was filed on the same day as the present application; and this application is related to U.S. patent application Ser. No. TBD, entitled LASER SURGICAL APPARATUS, to inventors Shlomo Assa, Steve J. Meyer and John Stine, which application was filed on the same day as the present application. The disclosures of the above two applications are incorporated herein by reference in their entirety.

### BACKGROUND

This specification relates to dental surgical lasers and, more particularly, to a disposable hand piece which carries the laser delivery optics and provides for fine mist for cooling of the treatment area.

In dental procedures, it is frequently desirable to remove portions of tooth enamel and dentin, and in certain cases, portions of gum tissue, in an accurately controlled manner and there has been a growing interest in the use of laser radiation for performing such procedures. The use of laser radiation is attractive because, particularly with the aid of optical delivery systems, such radiation can be focused to a very small area and is thus compatible with the dimensional scale of dental procedures. Moreover, laser radiation procedures can be performed without recourse to an anesthetic.

Laser use in dental enamel surgery was reported as early as 1964 using a ruby laser. Although such reports indicated that lasers could be used on dental hard tissue, lasers have not generally been used clinically until recently for surgical processes, including drilling teeth, because of the large amount of damage to nearby tissue that is often associated with such drilling. Pulsed eximer lasers as well as lasers producing infrared radiation have, however, been used recently for soft tissue and bone ablation due to the fact that these types of lasers have been found to do less damage than previous lasers.

The enamel and dentin of a tooth include, as one component, hydroxyapatite, which is in amorphous form in the dentin and crystalline form in the enamel. These portions of a tooth additionally include organic tissues and water, but have no vascular system. Healthy dentin is in mineralized form, while dentin which has experienced decay is in demineralized form. Dentin has a relatively high percentage of organic tissue, around 40 percent, and also a high percentage of water. These percentages increase considerably in decayed dentin.

Tooth pulp and the gum surrounding the teeth consist of vascularized organic tissue containing both hemoglobin and water. Each of these components has a different response to laser radiation. Moreover, it has been found, that hydroxyapatite absorbs laser radiation in the wavelength ranges of 9-11 μm., such as produced by CO₂ lasers, and also in the wavelength range 0.5-1.06.mu., which includes the wavelength that can be produced by a Nd:YAG laser.

While a particular wavelength may inherently have a cutting effect on enamel or dentin, it has been found that the practical utilization of radiation at such a wavelength for dental procedures is highly dependent on the form in which the radiation is applied, with respect to energy level, pulse duration and repetition rate. Specifically, efforts to apply such radiation in the form of high energy pulses of short duration have been found to produce a highly localized temperature increase, resulting in differential thermal expansion which can cause mechanical damage to the tooth as well as vascular damage to pulp tissue. Conversely, low energy pulses of long duration cause a more widespread heating of the tooth which results in patient discomfort as well as pulp damage due to heating.

The trend today is to use minimally invasive procedure that can repair tooth decay early, while minimizing patient's discomfort. Lasers have proved efficient and precise in other industrial field, promising potentially to better support the current trends.

Another important trend in medical technology in general, and in dental treatment in particular, is the use of selective area to be treated. The use of computerized means to distribute laser energy is applied in many cosmetic surgery applications today, and can be utilized similarly for dental treatments.

New detection tools for early detection of tooth decay are spreading fast in the dental sector, including tools and means to generate digital image of dental features, including but not limited to individual teeth or a portion of a tooth, all the way to entire oral cavity.

Dental procedure requires that all surgical instruments will be sterile, to avoid any cross contamination between patients and also to the treating dentist. Typically, all instruments will be sterilized using an autoclave. The need for sterilization of every dental hand piece creates a challenge for hand piece design and construction. Assemblies that are constructed of mechanical and optical components often trap water particles after autoclave sterilization, which is an undesired effect, and adversely affects the functionality. Over a period of time, water vapor residue will sometimes damages the components. Hospitals and outpatient clinics who use laser hand pieces for other medical procedures use gas sterilization or Gamma radiation sterilization methods. However, such tools are typically not available in the dentist's operatory.

Sharon et al. (U.S. Pat. No. 3,865,113) patented a laser beam manipulator device, particularly useful as a surgical scalpel, which includes a tube connectable at one end to a movable conduit down which a laser beam propagates and a beam targeting member carried by the tube at its opposite end. The manipulator device includes a lens that focuses the laser beam to a point in a plane substantially passing through the tip of the beam targeting member at right angles to the longitudinal axis of the tube. To permit viewing of the working area at the focal point of the laser beam while the device is being manipulated, at least a portion of the tip of the beam targeting member either is removed or is made of a transparent material.

Tanner (U.S. Pat. No. 4,517,974) patented disposable hand piece for attachment at the end of a laser catheter assembly. The hand piece is longitudinally tapered and is generally wedge-shaped in its width so as to form a tool which can be comfortable grasped by a surgeon for purposes of more accurately controlling the laser beam applied through the laser catheter. The hand piece includes an internal switching mechanism which may be conveniently activated by pressing flexible panels positioned on the sides of the hand piece. The hand piece is constructed of inexpensive plastic materials and is designed to be disposable after a single use.

Durden, III (U.S. Pat. No. 3,825,004) patented a disposable electrosurgical cautery which functions in a dual capacity as a hollow sucker tube as well as a cauterizer, and is intended to be prepackaged in sterilized containers to be used once and disposed of. The cautery consists of an elongated metal electrode tube having an electrical conductor wire permanently connected to a prelimal portion, and together are encased in a plastic housing which serves as an insulating handle. The handle is of special sculptured or contoured configuration to provide for deft and positive use of the distally projecting probe or point of the cautery without chance of short circuits or burns through inadequate wire connections or poor insulation. In operation, blood from a surgical incision or other wound is drawn by vacuum through the barrel of the electrode tube, clear of the severed vessels, and a high frequency current is passed through the electrode to cauterize and prevent further bleeding of the vessels.

### SUMMARY

This specification describes technologies relating to dental surgical lasers and, more particularly, to a disposable hand piece which carries the laser delivery optics and provides for fine mist for cooling of the treatment area.

In general, one or more aspects of the subject matter described in this specification can be embodied in a laser hand piece which is sufficiently economical in its construction as to be completely disposable after each use, thus eliminating the need for time consuming and costly resterilization techniques. The disposable hand piece can be attached to the end of a laser apparatus to assist a dental surgeon in directing and operating the laser. The disposable hand piece can be provided for dental surgery and can include: an elongated hand piece having a generally tapered, slightly bent, wedge-shaped configuration which is configured and arranged to be comfortably held in the hand in a pencil-like manner. A center volume can be formed through the length of the hand piece into which the laser Optical Assembly is positioned. A pair of metal tubes can be inserted into the laser optical assembly and can be attached to a main housing positioned in the forward portion of the hand piece. The tubes facilitate compressed air and water to flow, and be mixed to form fine cooling mist. Additionally, the hand piece can include a built in spring latch in the forward portion thereof. The spring latch can be used to securely anchor the end of the laser Optical Assembly in the hand piece. The hand piece can include a reflector that reflects the laser beam 90° and directs it to the area to be treated.

Particular embodiments of the subject matter described in this specification can be implemented to realize one or more of the following advantages. A disposable hand piece can be provided that can be attached to the end of a laser apparatus to facilitate handling. The hand piece can include means to direct the laser beam and provide the precision for the dental procedure, and means to provide cooling media for the treated area. The entire hand piece can be designed to be easily and inexpensively fabricated such that it can be disposed of together with the entire internal components assembly after a single use. This can result in a hand piece that is more effective and can be used under a wider range of conditions since the use of disposable components can reduce the inventory of parts, and further reduce the dependence on sterilization, which can increase the cost and time for a laser surgical procedure. Moreover, the hand piece can improve Dentist productivity with laser surgery by reducing the strain and fatigue associated with treating patients.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1 is a perspective view of a laser hand piece constructed in accordance with the present invention assembled on the laser apparatus.

FIG. 2 is an exploded perspective view of the laser hand piece of FIG. 1.

FIG. 3 is a perspective view of a laser hand piece constructed in accordance with the present invention.

FIG. 4 is an exploded perspective view of the laser hand piece of FIG. 3.

FIG. 5 is an exploded perspective view of the laser Optical Assembly from FIG. 2 disassembled from the laser apparatus.

FIG. 6 is a schematic cross-section of the hand piece along a front center line.

FIG. 7 is a perspective view of the Optical Assembly tube placement.

FIG. 8 is a schematic layout of the compressed air and water mixing components, which form fine cooling mist.

Like reference numbers and designations in the various drawings indicate like elements.

### DETAILED DESCRIPTION

FIG. 1 is a perspective view of a laser hand piece constructed in accordance with the present invention assembled on the laser apparatus. A disposable hand piece 7 connects with an articulated arm 10 beam delivery part of the laser apparatus used to generate and direct the laser emission. The last three rotatable mirrors in joints 9 enable a free maneuvering of the hand piece assembly so that the dentist can move the hand piece and aim it properly to the oral cavity section that will be treated. The final part 8 of the laser apparatus is used to connect the hand piece 7 to the laser apparatus in a manner that will be explain further below.

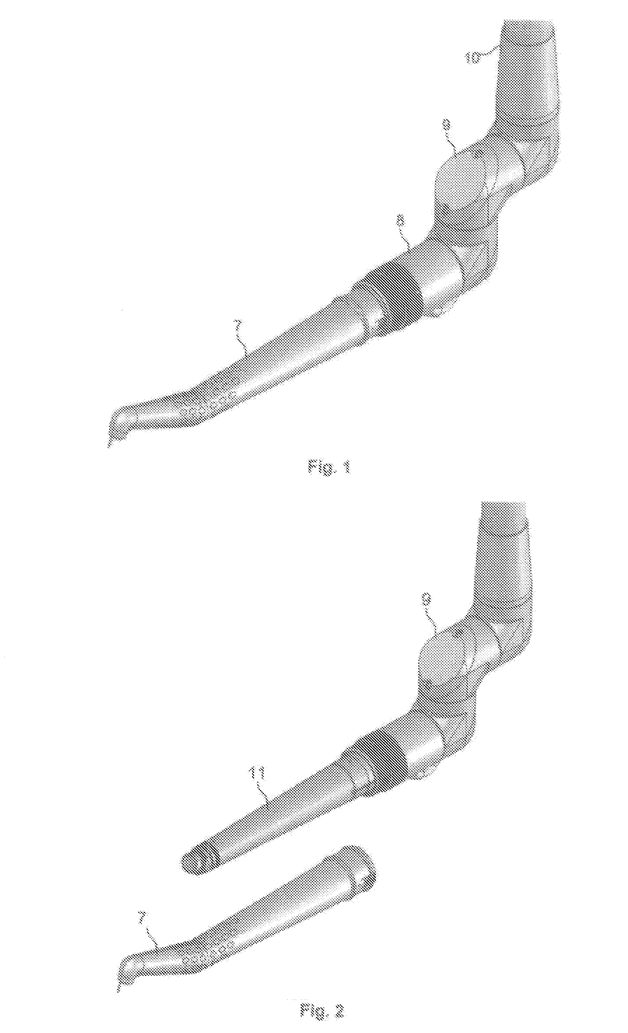

FIG. 2 is an exploded perspective view of the laser hand piece of FIG. 1. The disposable hand piece 7 had been removed, as the way it will be done at the end of the usage, before it is disposed of. An Optical Assembly 11 is mounted to the laser apparatus, and the disposable hand piece 7 attaches thereto.

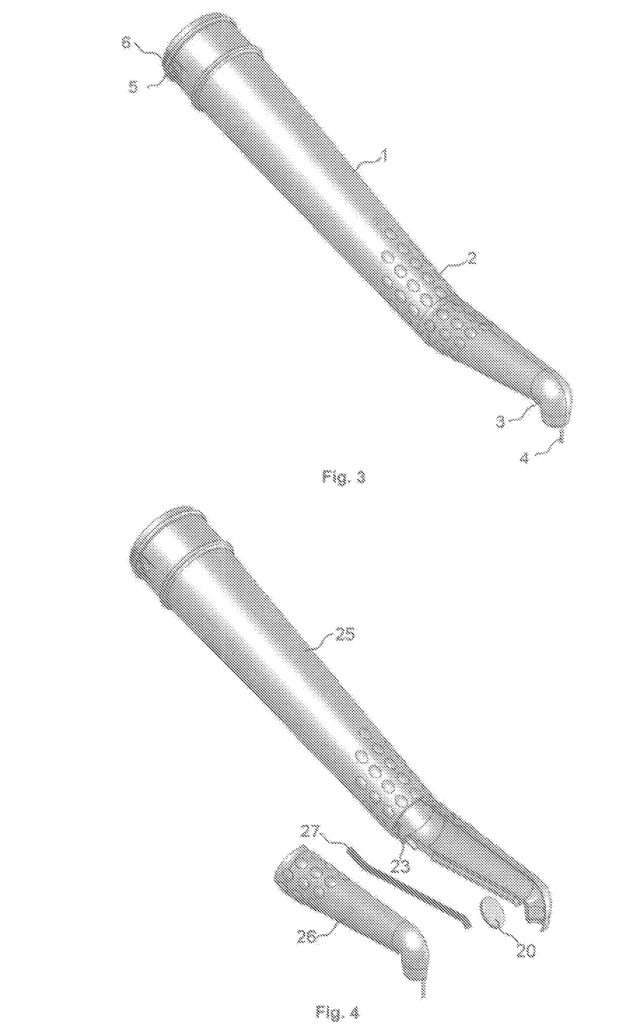

FIG. 3 is a perspective view of a laser hand piece constructed in accordance with the present invention. The hand piece main body 1 includes an elongated hand piece having a generally tapered, slightly bent, wedge-shaped configuration which is configured to be comfortably held in the hand in a pencil-like manner. Rubber molder bumps 2 can be provided for better grip and to avoid slipping in the hand while the hand piece is in use. An outlet 3 can be used to spray a fine air and water cooling mist aimed at the treatment area. A molded pointer 4 aimed to point the focus distance of the laser beam can also be provided for use in aiming the hand piece to the treatment area. The pointer 4 can be used to rest the dentist grip against the treatment area to improve the dentist aim and reduce the dentist fatigue. The pointer 4 can include molded marks every 1 mm, and the pointer 4 can be shortened by cutting a piece from the end. By shortening the pointer the focused beam can be moved to planes lower that the resting area, such that the laser can treat the bottom of pockets or craters. A molded button 5 can be pressed to release the hand piece locking mechanism from the Optical Assembly. By pressing the button 5, e.g., on both sides, the wedged latch 6 will bend and release the hand piece.

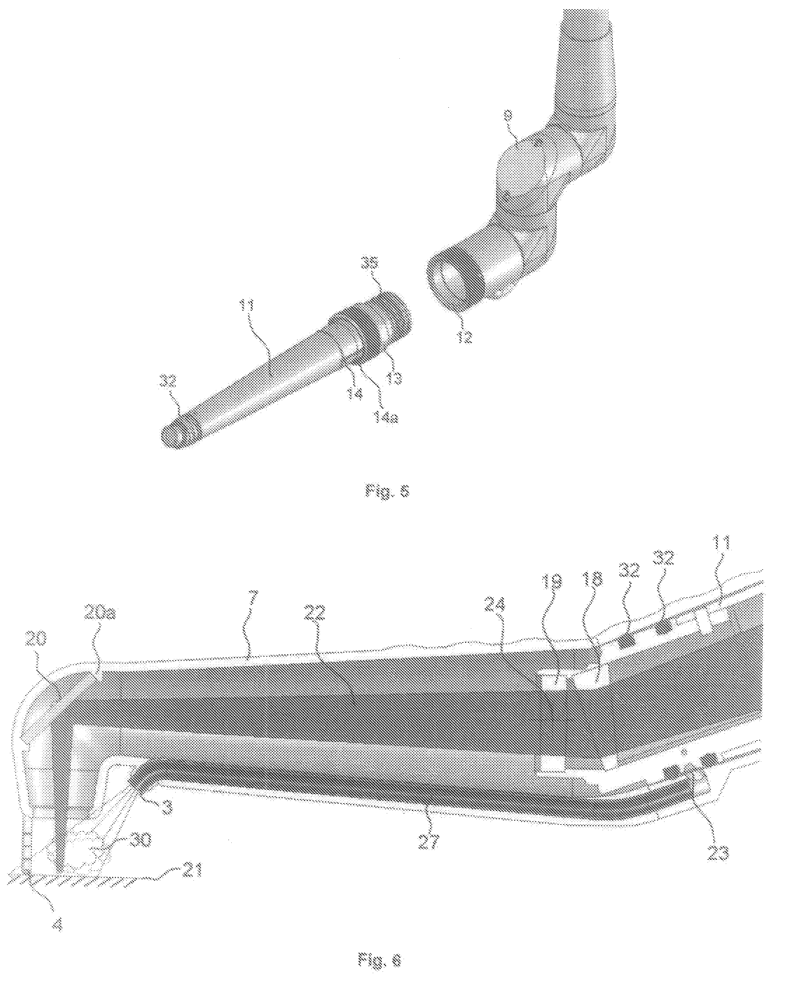

FIG. 4 is an exploded perspective view of the laser hand piece of FIG. 3. The primary object of the present invention is to provide a laser hand piece which is sufficiently economical in its construction as to be completely disposable after each use, thus eliminating the need for time consuming and costly resterilization techniques. The main hand piece part 25 can be made by injection mold from an inexpensive plastic grade. The molded part 25 can include (a) the main body that forms the center cavity, (b) locking latch 6 (FIG. 3), (c) locking release buttons, 5 (FIG. 3), (d) rubber over-mold bumps 2 (FIG. 3), (e) front half of the body 26, (f) cooling mist inlet hole 23, and (g) reflector mounting features 20a (FIG. 6). The front hand piece body cover part 26 can also be made by injection mold from an inexpensive plastic grade. Part 26 can include (a) main cover body, (b) rubber over-mold bumps, (c) molded pointer 4 (FIG. 3), and (d) reflector mounting features 20a (FIG. 6). Note that the front section 26 can be the entire distal end of the body, while functioning as a cover of one half of the distal end.

A reflector 20 can be made of #8 polished copper plate. The reflector can be cut from a solid plate to an elliptical shape fit to mount in a slot that is molded in both part 25 and part 26 to retain and mechanically hold the reflector in place. A preformed metal tube 27 can be inserted and secured in a mounting channel that is molded into part 25 and part 26. The tube provides the cooling mist to flow and spray the treatment area to provide for cooling. The plastic parts can be snapped into place and/or secured by inexpensive glue.

FIG. 5 is an exploded perspective view of the laser Optical Assembly from FIG. 2 disassembled from the laser apparatus. The Optical Assembly 11 us disconnected from the laser apparatus joints 9. The Optical Assembly 11 is mounted to the end of the articulated arm by engaging the male threads 13 to the female threaded ring 12 until the parts are locked. O rings 35 are to seal the water and air connection between the arm end and Optical Assembly 11. A guiding slot 14 can be cut in the metal body of the Optical Assembly 11 and can be used to orient the disposable hand piece in place by a molded pin that will slide into the slot 14. 14a is the locking ring that engages locking latch on part 6 of FIG. 3.

FIG. 6 is a schematic cross-section of the hand piece along a front center line, and showing the laser beam traced outline 22. An optical wedge 18 causes the laser beam to be tilted at 20°. Other angles can also be used, including angles that fall within the range of 5° to 30°. This tilt is part of the geometry used to enable easy reach to treat part of the oral cavity. The final focal lens 19 is used to focus the laser beam to a small spot at the treatment plain 21, that is at the end of the molded on pointer 4. The wedge 18 and the final focal lens 19 are mechanically secured in to the Optical Assembly 11.

The laser beam is reflected at 90° by a thin metal reflector 20. The reflector 20 can be cut from #8 thin polished copper plates. The reflector 20 is inserted and mechanically secured in place by molded on feature 20a design to secure the part in place.

The Optical Assembly 11 provides for the fine air and water mist in a way that will be detailed further below. The fine cooling mist flows from Optical Assembly 11 to the disposable hand piece 7 via molded on connecting hole 23. The mechanical connection between the Optical Assembly 11 and the disposable hand piece 7 can be sealed against air or water leaks using a pair of o rings 32. The mist is directed to the inserted metal tube 27, which is part of the disposable hand piece assembly, to aim the fine mist spray 30 at the treatment plane 21.

Another important objective that is part of this invention is that the disposable hand piece 7 and the Optical Assembly 11 are designed to provide for a collimated laser beam that is focused by the final focusing lens 19, forming a small spot size that increases the efficiency and effectiveness of the laser beam, and increases the effective usage depth of field. A smaller focused laser beam can increase the precision of the apparatus.

In some implementations, the operating parameters are: (a) laser beam 22 is dia. 5.0 mm is diameter, (b) the focal distance of final focusing lens 19 is 50 mm, (c) the laser beam is a CO₂ laser emitting wavelength of 9.3 μm, (d) laser beam quality factor is M Square=1.2, (e) the spot size is 142 μm, and (f) the effective working depth of field is ±1.4 mm.

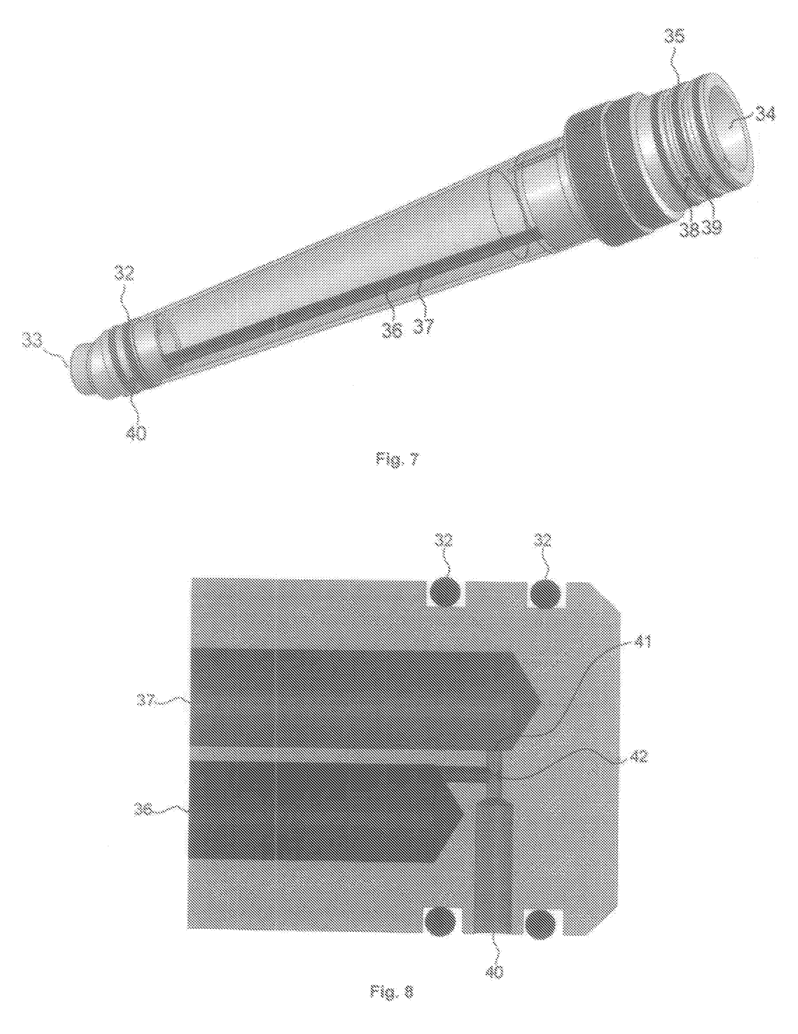

FIG. 7 is a perspective view of the Optical Assembly tube placement. O Rings 35 seal the connection from leaks of air or water. Compressed air is provided to hole 39, and the compressed air flows to tube 37 towards the end 40 of the Optical Assembly 11. Similarly, water will be provided to hole 38, the water will flow in tube 36 to the end 40 of optical assembly 11. The main space in the center of the Optical Assembly is for the laser beam that enters at 34 and will exit at 33.

FIG. 8 is a schematic layout of the compressed air and water mixing components, which form fine cooling mist. Compressed air from tube 37 flows to the restricted flow area 41. Similarly, the water from tube 36 flows to the restricted flow area 42. Restricted areas 41 and 42 are connected. The compressed air flowing velocity will increase at 41 due to the reduction of air flow crossection, causing the static pressure to reduce, and to suck the water and mix it with air to form the fine mist that is provided to hole 40.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a computer-readable medium. The computer-readable medium can be a machine-readable storage device, a machine-readable storage substrate, a memory device, or a combination of one or more of them.

While this specification contains many implementation details, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Thus, particular embodiments of the invention have been described, but other embodiments are within the scope of the following claims.

What is claimed is:

1. A disposable hand piece for controlling and directing the application of a laser beam produced by a laser powered by an electrical power source and transmitted through an optical beam delivery connected to said laser and encased in a sheath and mounted in a bore that extends through at least a portion of the disposable hand piece when attached, the disposable hand piece comprising:
an elongated conic and bent body having a proximal end and a distal end, the body comprising
a main portion having a monolithic conic tube shape that also defines an opening at the distal end, the main portion including bumps for improved grip, and
a cover portion having a formed edge that aligns with the main portion to cover the opening and form the body, the cover portion including bumps for improved grip;
a mechanism to anchor the hand piece to the optical beam delivery encased by the sheath;
a reflector mounted in the distal end between mounting features; and
a tube mounted to the body.

2. The disposable hand piece of claim 1, wherein the main portion is conic and straight.

3. The disposable hand piece of claim 1, wherein the laser beam exits straight in line with the bore.

4. The disposable hand piece of claim 1, wherein the laser beam is focused at a final lens secured to the hand piece in the optical beam delivery.

5. The disposable hand piece of claim 4, wherein the laser beam is scanned in two axes to form a pattern to cover an area with laser focused energy.

6. The disposable hand piece of claim 5, wherein the pattern is preprogrammed.

7. The disposable hand piece of claim 1, wherein the main portion and the cover portion of the hand piece are made of injection molded thermoplastic material.

8. The disposable hand piece of claim 1, wherein the main portion and the cover portion of the hand piece are made of plastic molding or plastic forming of a thermoset plastic material.

9. The disposable hand piece of claim 1, wherein the main portion and the cover portion of the hand piece are fabricated of metal by any form of casting or CNC (Computer Numerically Controlled) machining.

10. The disposable hand piece of claim 1, wherein the bumps for improved grip are made of the same material as the main portion and the cover portion of the hand piece.

11. The disposable hand piece of claim 1, wherein the reflector is cut from #8 polished thin copper plate.

12. The disposable hand piece of claim 1, wherein the tube comprises a preformed metal tube.

13. The disposable hand piece of claim 12, wherein the preformed metal tube is mounted outside of the body.

14. The disposable hand piece of claim 12, wherein the preformed metal tube is in a cavity defined within the distal end of the body.

15. The disposable hand piece of claim 1, wherein the bent body is bent at an angle between ten and thirty degrees.

16. The disposable hand piece of claim 1, wherein the laser beam is collimated.

17. The disposable hand piece of claim 16, wherein the laser beam provided from a fiber optics assembly.
